(19) United States
(12) Patent Application Publication
(10) Pub. No.: US 2024/0365762 A1
(43) Pub. Date: Nov. 7, 2024

(54) APPARATUS AND METHOD FOR BREEDING INSECTS IN A HIGH-BAY STORE

(71) Applicant: Alpha-Protein GmbH, Bruchsal (DE)

(72) Inventor: Rohi Shalati, Karlsruhe (DE)

(21) Appl. No.: 18/573,612

(22) PCT Filed: Jun. 9, 2022
(86) PCT No.: PCT/DE2022/100435

§ 371 Date: Dec. 22, 2023

(30) Foreign Application Priority Data
Jul. 2, 2021 (DE) 10 2021 117 134.2

(51) Int. Cl. A01K 67/033; A23D 9/00; A23J 3/04; A23K 10/20; A23K 20/147; A23K 20/158; A61K 8/98; A61Q 19/00; B08B 6/00; B65D 85/50

(52) U.S. Cl. CPC A01K 67/033; A23D 9/00; A23J 3/04; A23K 10/20; A23K 20/147; A23K 20/158; A61K 8/987; A61Q 19/00; B08B 6/00; B65D 85/50; A61K 2800/10

(57) ABSTRACT

The object of the invention is to allow insects to be reared in a high-rack warehouse in an automated, resource-saving and efficient manner.

The invention presented herein achieves this object in that a supply portal is arranged on the at least one high rack, to which a storage logistics facility can transport the boxes stored in a stackable manner in the at least one high rack, wherein the state of the box contents within the boxes transported from the storage logistics facility to the supply portal can be determined by means of the supply portal and the insects can be supplied by means of the supply portal and/or ejected when ready for harvest.

### REFERENCE TO PENDING PRIOR PATENT APPLICATIONS

This patent application is a 371 national stage entry of pending prior International (PCT) Patent Application No. PCT/DE2022/100435, filed 9 Jun. 2022 by Alpha-Protein GmbH and Rohi Shalati for APPARATUS AND METHOD FOR BREEDING INSECTS IN A HIGH-BAY STORE, which patent application, in turn, claims benefit of German Patent Application No. DE 10 2021 117 134.2, filed 2 Jul. 2021.

The two (2) above-identified patent applications are hereby incorporated herein by reference.

### FIELD OF THE INVENTION

The invention relates to an apparatus for rearing insects in a high-rack warehouse, comprising a storage region having at least one high rack and a storage logistics facility, wherein boxes can be stored in the at least one high rack in a stackable manner and the boxes can be transported within the storage region by means of the storage logistics facility, wherein insects are located in the boxes. The invention also relates to a method for rearing insects in a high-rack warehouse using an apparatus according to the invention.

### BACKGROUND OF THE INVENTION

An apparatus for rearing insects is known, for example, from the German patent DE 10 2019 121 102 B3. The apparatus allows insects to be reared automatically, in which a stack of boxes is rotated by means of a portal according to a paternoster principle and the insects located within the boxes are supplied by a supply module.

Furthermore, the international patent application WO 2016/153340 A2 discloses an apparatus and a method for rearing insects. The apparatus comprises
a spawning region in which there is a plurality of spawning containers adapted to receive or have received adult insects including mother insects and insect food, wherein the plurality of spawning containers is stacked in one or more stacks of spawning containers;
a plurality of spawning structures, wherein at least one spawning structure is adapted to be or is provided in each spawning container in the spawning region, wherein the mother insects will lay their eggs in these spawning structures, such that the spawning structure or a part of the spawning structure holds the eggs, wherein the spawning structure or part thereof can be removed from the spawning container, wherein the adult insects remain in the spawning container;
a hatching region in which the eggs will hatch, wherein the hatching region is adapted to receive the spawning structure or part thereof holding the eggs and is removable from the spawning containers, allowing harvesting of the baby larvae;
a food delivery system for delivering food to the spawning containers;
a spawning structure handling region comprising a spawning structure handling system adapted to remove the spawning structure or the part thereof holding the eggs from the spawning container and to transport the spawning structure or the part thereof to the hatching region and provide an empty spawning structure or part thereof in each spawning container, wherein the spawning structure handling region preferably is also adapted to clean the spawning structures or parts thereof;
and a container handling system for transporting the spawning containers between the spawning region and the spawning structure handling region.

Furthermore, from the international patent application WO 2016/166471 A1, a farm for raising insects is previously known, wherein the invention relates to a facility for rearing insects, comprising a first region in which the insects to be reared are stored in containers while they grow and a second region comprising at least one station configured to perform a rearing-related task on the insects in a container or on the container. The containers are grouped in the first zone into sets of palletized containers referred to as base units. The first zone comprises pallet tracks in which the base units are arranged. The first zone is also equipped with an automated apparatus configured to move the base units between the first zone and an interface having the second zone.

Furthermore, from the international patent application WO 2014/171829 A1, a method and a system for rearing insects using a large number of individual boxes is previously known, in which at least part of each box is filled with a substrate containing feed and immature phases of insects. In addition, a climate room is provided in which the boxes are housed and which has a ventilation system. A conveyor system is included in the system to retrieve and return the boxes to and from the climate room. Both an observation system for obtaining observations, including data and measurements, and a feed station downstream thereof for the starting material are arranged along the conveyor system.

Also known from international patent application WO 2017/223096 A1 is an autonomous food delivery platform configured to navigate through a facility and deliver insect food to multiple insect habitats located within the facility. In some cases, the food delivery platform may be configured to deliver the feed to multiple insect habitats at substantially the same time.

### SUMMARY OF THE INVENTION

Proceeding from this prior art, the object of the invention is to provide an automated, resource-saving, and efficient rearing of insects in a high-rack warehouse. This object is achieved with an apparatus according to independent claim 1. This object is further achieved with a method according to the independent claim 38. This also results in an oil according to the features of product claim 51, a flour according to the features of product claim 52, and a use of the flour and the oil according to the features of use claims 53 and 54. Advantageous embodiments of the invention can be found in the dependent claims.

In one aspect, the invention relates to an apparatus for rearing insects in a high-rack warehouse, comprising a storage region having at least one high rack and a storage logistics facility, wherein boxes can be stored in the at least one high rack in a stackable manner and the boxes can be transported within the storage region by means of the storage logistics facility, wherein insects are located in the boxes. So that the boxes to be transported only have to cover the shortest possible distances, among other things, the apparatus according to the invention provides that a supply portal is arranged on the at least one high rack, to which portal the storage logistics facility can transport the boxes stored in a stackable manner in the at least one high rack, wherein the state of the box contents within the boxes transported from the storage logistics facility to the supply portal can be determined by means of the supply portal and the boxes can be supplied by means of the supply portal and/or the boxes can be ejected when the insects are ready for harvest. The state of the box contents can be determined, for example, by labeling each box, wherein the labeling is read out by the supply portal and, based on the associated data, for example the duration of the storage period in the high-rack warehouse, the supply portal can decide whether and in what form the box filled with insects is supplied and/or ejected. Due to the short distances to be covered by the boxes from the storage logistics facility, an increased supply of the growing insects is made possible. In this way, an optimized rearing of the insects located within the boxes can ultimately be ensured.

The storage logistics facility can be a storage and retrieval device, a shuttle, or a flow rack for transporting the boxes from the at least one high rack to the supply portal.

The boxes can also contain not only the insects to be reared, but also insect skins, insect eggs, feces, feed, substrate, and other box contents, which ultimately also have an impact on the successful and efficient rearing of the insects. Furthermore, the boxes can be filled with different species of insects as well as with different stages of the insects. Depending on the type of insect, in particular grasshoppers, there is no stacked storage in the high rack, since the type of box to be used is not suitable for stacking.

In a first advantageous embodiment, the supply portal has at least one buffer region in which the boxes transported from the storage logistics facility to the supply portal can be temporarily stored. As a result, the efficiency can be increased further as a result.

In order to be able to determine in particular whether the optimal climate or the optimal conditions for rearing prevail in the boxes, the apparatus provides in a further advantageous embodiment that the supply portal has at least one detection device with which the parameters required for insect rearing, in particular humidity and temperature, can be determined inside a box. As a result, a more efficient rearing of the insects can be ensured, since the qualitative, physiological, and pathological conditions of the box contents can be determined. In another advantageous embodiment, the detection device has at least one sensor and/or an apparatus for recording and/or data transmission, in particular a camera, in order to be able to determine the corresponding parameters or the state of the box contents, in particular the insects, inside a box. In particular, the use of a camera makes it possible to determine an overall picture of the state of the insects and the remaining box contents within a box.

In an advantageous embodiment of the apparatus, the parameters determined by means of the detection device, which ultimately reflect the state of the box contents, in particular the insects, in one of the boxes, can be compared with a reference database, so that the relevant state of the box contents and/or the degree of maturity of the insects can be determined. The data obtained in this way by the detection device can also be used to set up a reference database and, by comparing the data stored in the reference database with currently determined parameter data, for machine learning of the detection device. Ultimately, the data obtained in this way can preferably be used for the automated optimization of the production parameters, in particular to improve the formulation and dosage of the feed. As a result, a further optimization of the rearing of insects can be achieved.

Furthermore, in an advantageous embodiment of the apparatus, the supply portal has at least one supply module with which the insects within the boxes transported by the storage and retrieval device into the supply portal can be supplied with supply materials. In particular, the supply module adds feed and substrate to the boxes to encourage the growth of the insects located within the boxes.

In order to be able to remove unwanted by-products of the insects, in particular insect skins, feces, and uneaten food residues, wherein the latter spoils and thus poses a potential risk of the insects becoming ill, the apparatus provides in a further advantageous embodiment that the supply portal has at least one cleaning module. With this cleaning module, the supply portal can remove the rest of the box contents, in particular insect skins, feces, and feed residues, from the box and thus also contribute to an increased growth rate of the insects. In another advantageous embodiment, the cleaning module has at least one suction apparatus, with which the rest of the box contents can be removed from a box by suction or air classification. The removal of the rest of the box contents, in particular the insect skins and the feces of the insects, can also be carried out by at least one electrical device present on the cleaning module, with which the corresponding other box contents can be removed from a box by electrostatics.

Furthermore, in an advantageous embodiment, the apparatus provides that the apparatus according to the invention has at least one hatching region in order to optimize the automated rearing of insects in a high-rack warehouse. Hatching processes take place in this hatching region; in particular insect eggs and/or pupae laid by reproducing insects can hatch in this region. However, insect eggs or pupae obtained from other sources can also be hatched in the hatching region. The insect eggs and/or pupae can be arranged in boxes, flat boxes, and/or flat trays. As soon as the larvae and/or insects in particular have hatched from the insect eggs and/or pupae, they can be processed further in a pupae processing region in a further advantageous embodiment of the apparatus. The insects intended for rearing can thus be reproduced independently and then transferred, for example by a suitable transport system and the storage logistics facility available in the storage region, to the at least one high rack, on which a supply portal is arranged, for further rearing.

In order to achieve the optimal conditions for incubating the insect eggs and/or pupae and thus ultimately an increased number of hatched larvae and/or insects, the apparatus provides in a further advantageous embodiment that the hatching region has an incubator for incubating the insect eggs and/or pupae. In this way, it can be ensured that the optimal conditions for the hatching of the larvae and/or insects from the insect eggs and/or pupae prevail, resulting in a further increase of the insect rearing efficiency.

Furthermore, the invention provides for the apparatus in an advantageous embodiment that it has at least one separating device, with which the insect eggs and/or pupae or the larvae and/or insects hatched in the hatching region, in particular in the incubator, as well as insect pupae processed in the pupae processing region, larvae, or insects can be separated from the rest of the box contents. It is thus possible to separate any eggs and/or pupae that have not yet hatched from the larvae and/or insects that have already hatched from the insect eggs and/or insect pupae and remove the larvae and/or insects that have hatched from the hatching region or pupae processing region for further rearing into the at least one high rack in the storage region and to transfer the possibly not yet hatched eggs or pupae back to the hatching or pupae processing region for further treatment. Residues of the larvae and/or insects that have hatched from the insect eggs and/or pupae, as well as the remaining eggshells or pupae, can also be removed by the separating device in the hatching or pupae processing region.

In order to prepare the containers for filling with insect eggs or to be able to care for the larvae and/or insects hatched from the insect eggs and/or insect pupae in the incubator and in order to, for example, allow a means of transport or a conveyor system to cover the shortest possible distances, the apparatus provides in a further advantageous embodiment that it has at least one supply device with which the container filled with insect eggs and/or insect pupae can be filled with corresponding supply materials. This has the advantage that all the steps required for complete reproduction, starting with the incubation of the insect eggs and/or insect pupae through to the (initial) care of the hatched larvae and/or insects and placing them in a box for further rearing, can be implemented within a region.

If the insects located within the boxes are ready to be harvested or have reached the degree of maturity required for further processing, they must be unstacked within the at least one high-rack stackable box, the box contents removed from the boxes, and the boxes cleaned. In order to achieve this object, the apparatus provides in an advantageous embodiment that it has at least one emptying region in which the aforementioned steps can be carried out.

In this case, in a further advantageous embodiment, the emptying region has at least one emptying device with which the unstacking of the boxes and the removal of the box contents from the boxes can be carried out. This means that the stackable boxes filled with insects can be emptied quickly and gently.

In order to be able to reuse the boxes unstacked and emptied in the emptying region, the apparatus provides in another advantageous embodiment that the emptying region has at least one cleaning device with which the emptied and unstacked boxes can be cleaned.

So that the box contents emptied from the boxes can be separated in the emptying region, the apparatus provides in an advantageous embodiment that there is at least one separation region in which the box contents emptied from the boxes can be separated into products, intermediate products, and other box contents. In particular, products can be insects of a desired size that are ready for harvest, intermediate products can be insects that are not yet fully mature, and other box contents, for example feed residues, feces, and insect skins. In another advantageous embodiment, the box contents emptied from the boxes can be transported to the separation region in the form of bulk material.

In another advantageous embodiment, the apparatus provides that the boxes cleaned and emptied by means of the cleaning device in the emptying region can be stored in at least one buffer region and made available for further use.

Furthermore, in an advantageous embodiment, the apparatus provides that it has at least one filling region in which the boxes can be filled with feed, insects, insect eggs, substrates or a combination thereof. The filling region is used essentially for the initial stocking or initial filling or refilling of the boxes with the materials essential for rearing, in particular substrate, insect eggs, larvae and feed. Using the filling region, boxes can also be filled for the first time with insects, insect larvae, or newly hatched insects intended for rearing and supplied with the appropriate materials for further rearing. The filling region preferably has at least one supply portal for the initial stocking or refilling of the boxes intended for rearing. The filling region particularly preferably has a supply portal for at least one existing stage of the insects, in particular for pupae, newly hatched larvae, or beetles.

Furthermore, in an advantageous embodiment, the apparatus provides that the filling region has at least one filling line, in which the filling of the boxes takes place. The filling line can have different stations or modules that equip the box to be filled with the necessary materials.

So that the boxes stored in the storage region in a stackable manner in the at least one high rack and which can be transported by the storage logistics facility can be transported out of and into the storage region, the apparatuses provide in an advantageous embodiment that it has at least one conveyor system with which the boxes can be transported out of and into the storage region.

In another advantageous embodiment, the apparatus provides that the conveyor system is entirely or partially a driverless transport system. A driverless transport vehicle is a floor-bound conveyor with its own drive that is automatically controlled and guided without contact. These driverless transport vehicles are usually used to transport materials, namely to pull or carry goods with active or passive load handling devices, but not to transport people. Such transport systems substantially consist of one or more driverless transport vehicles, a master controller, a device for determining the location and detecting the position, a device for data transmission, and an infrastructure and a peripheral device.

Furthermore, in another advantageous embodiment, the apparatus provides that it has at least one delivery region to which materials, in particular feed, raw materials for feed, substrate, or any other materials necessary for rearing insects, can be delivered.

At least one feed raw material silo can preferably be arranged at the delivery region, in which, among other things, raw materials for feed production can be stored and made available for further use.

In a further advantageous embodiment, the apparatus can also have at least one product silo in which a product obtained from the rearing, for example live insects for use as animal feed, can be stored.

In order to ensure optimal growth conditions for the insects within the boxes stored in at least one high rack so that they can be stacked, the apparatus provides in a further advantageous embodiment that the storage region has at least one air conditioning device with which the air quality within the storage region is regulated. Air quality is understood to mean, among other things, the temperature, the humidity, and the concentration of particles in the air. The optimal conditions for efficient growth of the insects can thus be set by means of the air conditioning device.

It is also advantageous if within the storage region there is access to the at least one supply portal arranged on the high rack, so that the apparatus provides in an advantageous embodiment that within the storage region on the at least one supply portal arranged on the high rack at least one platform for maintenance, for manual intervention, and for manual loading of the supply portal is available.

Furthermore, in another advantageous embodiment, the apparatus provides that the storage region is designed in a silo design, so that the at least one high rack is used as a supporting element for at least one outer facade. As a result, material savings can be achieved, which in turn can ultimately ensure resource-saving and efficient rearing of insects. In a further advantageous embodiment, the apparatus provides that the at least one high rack used as a supporting element and outer facade has insulation with a vapor barrier, so that the storage region is shielded from the outside. In this way, it can be ensured in particular that a change in the climatic conditions outside has little or no effect on the conditions prevailing inside the storage region.

In a second aspect, the invention relates to a method for rearing insects using an apparatus according to the invention, in which a stack of boxes is taken from at least one high rack and transported to a supply portal, wherein for each of the boxes transported into the supply portal the state of the box contents of the relevant box is determined by means of the supply portal and the box is ejected or supplied depending on the determined state of the box contents, wherein, after the supply, the respective boxes are transported back into the at least one high rack. The supply can be made dependent on the state of the box contents, in particular depending on the age, the population, the life stage of the insects, the batch, and other factors. In this way, an automated and efficient rearing of insects in a high-rack warehouse can be achieved.

In order to further increase the efficiency of the method for rearing insects in a high-rack warehouse, the method provides in an advantageous embodiment that the storage of the stackable boxes in at least one high rack is not stored according to a fixed place system, but according to the chaos principle or another optimized storage type, wherein the chaos principle is a so-called dynamic warehousing in which a part to be stored does not have a fixed storage location in the storage region, but is stored in any location that is not occupied at the time of storage. As a result, among other things, a leveling of the degree of machine utilization of the supply portals, the storage logistics facility, and better use of the storage region can be achieved, and new stackable boxes can be stored and changed more easily.

In a further advantageous embodiment, the method also provides that boxes transported to the supply portal are transported from this supply portal to another supply portal. This can be done, for example, by the storage logistics facility itself or by a means of transport suitable for this purpose. This is advantageous when the capacity of the supply portal to which the boxes were transported is fully utilized or the corresponding supply portal is defective or needs maintenance. In order to avoid downtime, these boxes are therefore transported to another existing supply portal, the capacity of which is not fully utilized, so that the boxes can be examined there accordingly and, if necessary, ejected, and/or supplied. As a result, faster processing of the boxes is ensured and downtimes are avoided.

Furthermore, the method provides that the substrate and/or feed is/are removed from boxes that are ready to be harvested, so that the insects can empty their intestines. This is particularly advantageous because it means that cleaning the raw product obtained from the insects can be avoided, since the raw product is thereby already almost completely freed from any excrements still present in the insects. The separation preferably takes place 1 to 8 days, particularly preferably 1 to 5 days, before the actual harvest, i.e., before the box is ejected from the storage region, for example by the supply portal or by the storage logistics facility for further processing. This is because the proportion of nutrients in the product, i.e. the insect, can be increased and the other components, for example water and excrement, can be reduced.

Furthermore, in another advantageous embodiment, the method provides that the boxes ready for harvest, ejected from the supply portal or the storage logistics facility, are unstacked and emptied. In an advantageous embodiment of the method, the emptied boxes can also be cleaned. Furthermore, the cleaned boxes can be stored and made available for further use, in particular for receiving intermediate products or for refilling.

The method also provides that the box contents originating from the emptied boxes are separated into products, intermediate products, and other box contents. In this case, products can be insects of a corresponding weight or size, wherein smaller insects or lighter insects are considered to be intermediate products which, in a further advantageous embodiment of the method, can be filled back into a box for further rearing. The other box contents, which may consist, for example, of dead insects, insect skins, residues of substrate and/or feed, feces, and other components, can be diverted separately as other box contents for further destruction or processing.

In order to be able to further process the insects now regarded as a product, the method provides, in another advantageous embodiment, that the products are either obtained as a living product or are inactivated and shredded. The inactivation can be implemented in particular by a sharp change in temperature, wherein this heating or cooling can take place in particular by blanching or freeze-drying. After inactivation, the product can be shredded, and the shredded product thus obtained can be fed to the further processing steps.

In a further advantageous embodiment, the method provides that the shredded product is degreased and an oil is formed from the product. In a further advantageous embodiment of the method, the degreased product obtained in this way can then be dried and ground, so that flour can ultimately be obtained.

In a further aspect, the invention provides for the use of a flour obtained from the method for feed for insect and animal rearing and for foodstuff, in particular in the form of protein powder.

Furthermore, in a further aspect, the invention provides the use of an oil obtained from degreasing for food and cosmetic products and for feed. For example, the oil can be used as a pure oil to prepare salad dressings or as a substitute for palm oil in food production. In addition, the use in cosmetic products, in particular as a substitute for palm oil, is also conceivable. Ultimately, the oil itself can be used or for the production of feed.

The invention is then explained in more detail using an embodiment.

### BRIEF DESCRIPTION OF THE DRAWINGS

In the drawings:

FIG. 1: is a schematic view of an apparatus for rearing insects in a perspective view.

### DETAILED DESCRIPTION OF THE PREFERRED EMBODIMENTS

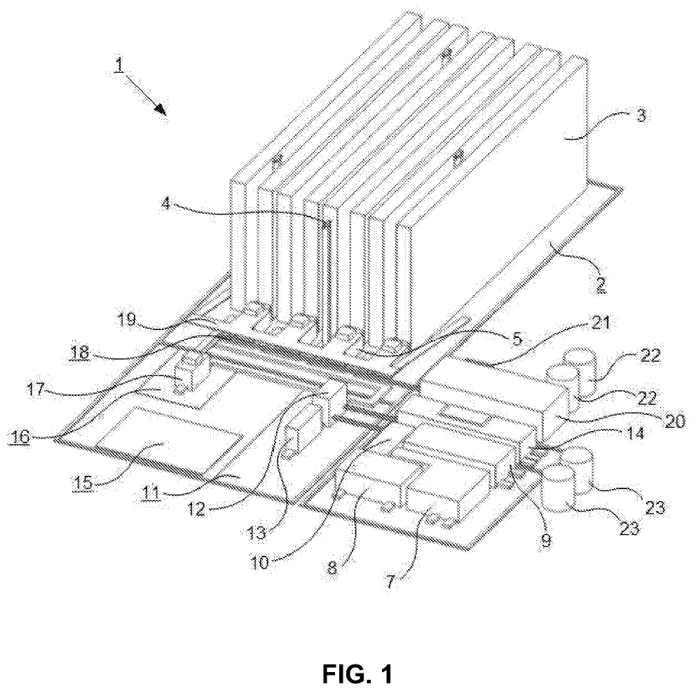

FIG. 1 is a schematic view of an apparatus for rearing insects in a perspective view. In the storage region 2, a plurality of high racks 3 are arranged in rows of two, wherein a storage and retrieval device 4 can be moved along the free spaces formed between the rows of two as a storage logistics facility and the storage and retrieval device 4 can transport boxes to and from the relevant high rack 3. In addition, the storage and retrieval devices 4 can move out of the high racks 3 and to the supply portals 5 arranged on every second high rack 3 and thus transport boxes from the high racks 3 into the supply portals 5 arranged on every row of two.

The boxes transported from the high racks 3 by means of the storage and retrieval devices 4 to the supply portals 5 arranged on the rows of two can be examined within the supply portals 5, wherein the supply portal 5 supplies the boxes with supply materials, in particular feed and substrate, depending on the state of the box contents, or in case a box ready for harvest can eject it from the storage region 2. Furthermore, boxes ready for harvest can also be ejected directly from the storage region 2 by means of the storage and retrieval device 4. This can happen, for example, due to an elapsed time interval based on empirical data. It is thus possible to eject the box contents from the storage region 2 by means of the storage and retrieval device 4 without checking the state of the box contents through a supply portal 5.

After a possible supply of the boxes through the supply portal 5, they can be removed from the supply portals 5 again by means of the storage and retrieval device 4 and brought back to the high racks 3 for further rearing, wherein the storage or accommodation of the boxes supplied by means of the supply portal 5 can take place, for example in accordance with the chaos principle or another form of optimized warehousing. The boxes supplied can also initially be temporarily stored in a buffer region present at the supply portal 5 until the storage and retrieval device 4 brings them back to the high rack 3.

In the event that the boxes are stored in the form of stacks or double stacks, it is conceivable that the supply portal 5 has the necessary device to unstack these stacks of boxes accordingly in order to carry out the appropriate examination and supply of the boxes.

In order to be able to intervene manually during the automated supply of the boxes through the supply portals 5, there is a platform 19 along the supply portals 5 arranged on the high racks 3, so that, for example, employees can manually take a sample from the boxes or from the supply materials or can perform an inspection.

For the initial or new stocking or refilling of boxes with insects and the corresponding materials required for breeding, the apparatus 1 has in this case a filling region 16 having an upstream supply portal 17. The filling region is in this case connected, by means of the conveyor system 18, to the delivery region 20 and the feed raw material silos 22 adjacent to the delivery region 20, in which silos delivered feed raw material for further processing or use are kept, as well as to the storage region 2 and to the separation region 14. For the delivery of any materials, in particular feed raw materials, the delivery region 20 has a truck ramp 21 in this case, which ensures easy unloading of a truck filled with the appropriate materials. Furthermore, there can be any equipment in the delivery region with which the delivered feed raw materials can be prepared or processed before they are then stored in the feed raw material silos 22 for further processing or use.

If boxes have been identified as ready for harvest in the supply portal 5, the supply portal 5 can eject these boxes or the boxes are ejected directly from the storage region 2 by the storage and retrieval device 4 based on the empirical assumption that they are now ready for harvest. The supply portals 5 and the storage and retrieval devices 4 can therefore both eject boxes that are ready for harvest from the storage region 2 and transport them to the emptying region 11 for further treatment by means of the conveyor system 18 that is also connected to the emptying region 11. After arriving in the emptying region 11, the boxes identified as ready for harvest can be unstacked and emptied there by means of the emptying device 12, and the emptied boxes can be cleaned by means of the cleaning device 13 arranged on the emptying device 12. However, cleaning does not have to take place. It is also conceivable to directly reuse the emptied boxes without prior cleaning. The boxes cleaned by the cleaning device 13 can then be kept in a buffer region 15 for further use, in particular for refilling or for filling insect eggs or insect pupae.

The box contents emptied by means of the emptying device 12 can then be transported by the conveyor system 18 from the emptying region 11 to the separation region 14. Once there, the emptied box contents can be separated into products, intermediate products, and other box contents, wherein intermediate products, for example insects that are not fully mature, can be transported back to the filling region 16 by means of the conveyor system 18 for restocking. The insects separated as a product, for example fully mature insects or insects with a certain degree of maturity, can then either be stored as a living product in product silos 23 or be kept available for further processing. The living products can also be processed further, in particular to obtain oil and flour. Such end products obtained from the products can also be stored in product silos 23 before they are transported to another location, for example by truck or goods train, for further processing or use. The other box contents separated from the intermediate products and products, in particular insect skins and insect feces, can be used, for example, for use as or for the production of fertilizer.

In addition, the apparatus 1 shown in FIG. 1 has a hatching region 7 and a pupae processing region 9 with a supply device 8 and a separating device 10, in which processing steps for optimizing the insects intended for rearing can be carried out automatically. Containers, in particular boxes, which are filled with insect eggs and/or insect pupae can be stored in the hatching region 7 and the insect eggs and/or insect pupae can be incubated under the optimum conditions within the hatching region 7 or pupae processing region 8. The hatching region 7 preferably has an incubator for this purpose. It is also conceivable that the containers filled with insect eggs and/or insect pupae are stored in the high rack 3. So that the larvae and/or insects hatched from the eggs and/or pupae in the hatching region 7 and/or pupae processing region or in the high rack 3 can be supplied, the apparatus 1 has a supply device 8 in which the hatched eggs and/or pupae can be supplied in particular with feed and substrate for optimal growth.

So that the larvae hatched from the eggs in the hatching region 7 can be transported and supplied, the apparatus 1 can also have a transport, supply, dosing, and handling device with which the newly hatched larvae and/or insects can be supplied, for optimal growth, with the necessary materials, in particular the right feed. The assembly with feed, substrate, or larvae portions for further rearing in the storage region 2 can take place either directly by the supply device 8 or in the filling region 16.

When the hatched larvae and/or insects have reached an appropriate stage for further rearing, they can be separated by the separating device 10 from any unhatched eggs or pupae, dead insects, and any other box contents. The young larvae or young insects separated in this way from any other box contents can then be treated in the pupae processing region 9 for further rearing or filled into boxes by being transported with the conveyor system 18 within the filling region 16 through the upstream supply portal 17 together with materials for further rearing and transported to the storage region 2 for further rearing.

An apparatus for rearing insects in a high-rack warehouse is thus disclosed above, which allows insects to be reared in an automated, resource-saving, and efficient manner.

### LIST OF REFERENCE SIGNS

1 Apparatus
2 Storage region
3 High rack
4 Storage and retrieval device
5 Supply portal
7 Hatching region
8 Supply device
9 Pupae processing region
10 Separating device
11 Emptying region
12 Emptying device
13 Cleaning device
14 Separation region
15 Buffer region
16 Filling region
17 Upstream supply portal
18 Conveyor system
19 Platform
20 Delivery region
21 Truck ramp
22 Feed raw material silo
23 Product silo

1. Apparatus (1) for rearing insects in a high-rack warehouse, comprising a storage region (2) having at least one high rack (3) and a storage logistics facility, wherein boxes can be stored in the at least one high rack (3) in a stackable manner and the boxes can be transported and/or ejected within the storage region (2) by means of the storage logistics facility, wherein insects are located in the boxes,
characterized in that a supply portal (5) is arranged on the at least one high rack (3), wherein the boxes are storable in the high rack (3) in a stackable manner and are transportable from the at least one high rack (3) to the supply portal (5) by means of the storage logistics facility, and the state of the box contents within the box are determinable by means of the supply portal (5), and the insects in the boxes are suppliable with the supply portal (5), and/or the boxes are ejectable.

2. Apparatus according to claim 1, characterized in that a box has box contents in the form of insects, insect skins, insect eggs, feces, substrate, and/or feed.

3. Apparatus according to claim 1, characterized in that the storage logistics facility is a storage and retrieval device (4), a shuttle, or a flow rack.

4. Apparatus according to claim 1, characterized in that the supply portal (5) has at least one buffer region in which the boxes transported from the storage logistics facility to the supply portal (5) can be temporarily stored.

5. Apparatus according to claim 1, characterized in that the supply portal (5) has at least one detection device with which the parameters required for insect rearing, in particular humidity and temperature, can be determined inside a box.

6. Apparatus according to claim 5, characterized in that the detection device has at least one sensor and/or an apparatus for recording and/or data transmission, in particular a camera.

7. Apparatus according claim 5, characterized in that the parameters determined by the detection device, which reflect the state of the contents in one of the boxes, can be compared with a reference database, so that in particular the relevant state of the box contents and/or the degree of maturity of the insects within a box can be determined.

8. Apparatus according to claim 7, characterized in that the data obtained by comparing the determined parameters with the reference database can be used for machine learning of the detection device.

9. Apparatus according to claim 8, characterized in that the data obtained can be used for the automated optimization of the production parameters, in particular the formulation and dosage of the feed.

10. Apparatus according to claim 1, characterized in that the supply portal (5) has at least one supply module with which the insects in a box can be supplied with supply materials, in particular feed and substrate.

11. Apparatus according to at least claim 1, characterized in that the supply portal (5) has at least one cleaning module with which parts of the box contents, in particular skins of insects, feces, and feed residues, can be removed from a box.

12. Apparatus according to claim 11, characterized in that the cleaning module has at least one suction apparatus with which the parts of the box contents to be removed can be removed from a box by suction or air classification.

13. Apparatus according to claim 11, characterized in that the cleaning module has at least one electrical device with which the part of the box contents to be removed can be removed from a box by electrostatics.

14. Apparatus according to claim 1, characterized in that it has at least one hatching region (7).

15. Apparatus according to claim 14, characterized in that the hatching region (7) has an incubator for incubating insect eggs and/or insect pupae.

16. Apparatus according to claim 1, characterized in that it has at least one pupae processing region (9).

17. Apparatus according to claim 14, characterized in that the apparatus comprises at least one separating device (10) with which the insect eggs and/or insect pupae or the larvae and/or insects hatched in the hatching region (7) and/or pupae processing region (9) and/or in the high-rack warehouse hatched larvae and/or insects can be separated from the rest of the box contents.

18. Apparatus according to claim, 14 characterized in that the apparatus comprises it has a supply device (10) with which the insect eggs, insect pupae, hatched larvae, and/or insects can be supplied with supply material.

19. Apparatus according to claim 1, characterized in that the apparatus comprises at least one emptying region (11) in which the boxes can be unstacked and the box contents can be removed from the boxes.

20. Apparatus according to claim 19, characterized in that the at least one emptying region (11) has an emptying device (12) with which the unstacking of the boxes and the removal of the box contents from the boxes can be carried out.

21. Apparatus according to claim 19, characterized in that the at least one emptying region (11) has at least one cleaning device (13) with which the unstacked and emptied boxes can be cleaned.

22. Apparatus according to claim 19, characterized in that at least one separation region (14) is present, in which the box contents emptied from the boxes can be separated into products, intermediate products, and other box contents.

23. Apparatus according to claim 22, characterized in that the box contents emptied from the boxes can be transported in the form of bulk material to the separation region (14).

24. Apparatus according to claim 19, characterized in that the apparatus comprises at least one buffer region (15) in which empty and/or cleaned boxes can be kept.

25. Apparatus according to claim 1, characterized in that the apparatus comprises at least one filling region (16) in which the boxes can be filled with feed, insects, insect eggs, or a combination thereof.

26. Apparatus according to claim 25, characterized in that the filling region (16) has at least one upstream supply portal (17) for filling the boxes.

27. Apparatus according to claim 26, characterized in that an upstream supply portal (17) is present for at least one stage of the insects.

28. Apparatus according to claim 25, characterized in that the filling region has at least one filling line in which the filling of the boxes takes place.

29. Apparatus according to claim 1, characterized in that the apparatus comprises at least one conveyor system (18) with which in particular the boxes or stacked or palletized stacks of boxes can be conveyed out of and into the storage region (2).

30. Apparatus according to claim 29, characterized in that the conveyor system (18) is wholly or partially a driverless transport system.

31. Apparatus according to claim 1, characterized in that the apparatus comprises it has at least one delivery region (20) to which materials, in particular feed for rearing the insects, can be delivered.

32. Apparatus according to claim 26, characterized in that at least one feed raw material silo (22) is arranged at the delivery region (20).

33. Apparatus according to claim 1, characterized in that it has at least one product silo (23) in which a product obtained from the rearing of the insects can be stored.

34. Apparatus according to claim 1, characterized in that the storage region (2) has at least one air conditioning device with which the air properties within the storage region (2) can be regulated.

35. Apparatus according to claim 1, characterized in that the storage region (2) on the at least one supply portal (5) arranged on the high rack (3) has at least one platform (19) for maintenance, for manual intervention, and for manual loading of the supply portal (5).

36. Apparatus according to claim 1, characterized in that the storage region (2) is designed in a silo design, so that the at least one high rack (3) is used as a supporting element for at least one outer facade.

37. Apparatus according to claim 36, characterized in that the at least one high rack (3) used as a supporting element for at least one outer facade has insulation with a vapor barrier, so that the storage region (2) is shielded from the outside.

38. A method for rearing insects in a high-rack warehouse, the method comprising:
providing apparatus (1) for rearing insects in a high-rack warehouse, comprising a storage region (2) having at least one high rack (3) and a storage logistics facility, wherein boxes can be stored in the at least one high rack (3) in a stackable manner and the boxes can be transported and/or ejected within the storage region (2) by means of the storage logistics facility, wherein insects are located in the boxes,
characterized in that a supply portal (5) is arranged on the at least one high rack (3), wherein the boxes are storable in the high rack (3) in a stackable manner and are transportable from the at least one high rack (3) to the supply portal (5) by means of the storage logistics facility, and the state of the box contents within the box are determinable by means of the supply portal (5), and the insects in the boxes are suppliable with the supply portal (5), and/or the boxes are ejectable;
taking a stack of boxes from at least one high rack (3) and transporting the stack of boxes to a supply portal (5), wherein for each of the boxes transported into the supply portal (5) the state of the box contents of the relevant box is determined by means of the supply portal (5) and the boxes are ejected or supplied depending on the determined state thereof,
wherein, after the boxes are supplied, the respective boxes are transported back into the at least one high rack (3).

39. The method according to claim 38, characterized in that the boxes transported into and out of the at least one high rack (3) are stored in the at least one high rack (3) according to a chaos principle or another form of storage optimization.

40. The method according to claim 38, characterized in that the boxes transported to the supply portal (5) are transported from this supply portal (5) to another supply portal.

41. The method according to claim 38, characterized in that the substrate and/or feed is/are removed from boxes that are ready to be harvested so that the insects empty their intestines.

42. The method according to claim 41, characterized in that the removal of the substrate and/or feed is carried out 1 to 5 days before the ejection of the box ready for harvest.

43. The method according to claim 38, characterized in that ejected, harvest-ready boxes are unstacked and emptied.

44. The method according to claim 43, characterized in that the unstacked and emptied boxes are cleaned.

45. The method according to claim 43, characterized in that the boxes are stored and kept available for further use.

46. The method according to claim 43, characterized in that the emptied box contents is separated into products, intermediate products, and other box contents.

47. The method according to claim 46, characterized in that the intermediate products are filled into boxes for further rearing.

48. The method according to claim 46, characterized in that the product is preserved as a living product or inactivated and shredded.

49. The method according to claim 48, characterized in that the shredded product is degreased and an oil is formed.

50. The method according to claim 49, characterized in that the degreased product is dried and ground to form flour.

51. Oil produced by a method according to claim 49.

52. Flour produced by a method according to claim 50.

53. Use of a flour according to claim 52 as feed for animal breeding and for food, in particular as a protein-containing powder.

54. Use of an oil according to claim 51 for food and feed as well as cosmetic products.
